(19) United States
(12) Patent Application Publication
(10) Pub. No.: US 2025/0044206 A1
(43) Pub. Date: Feb. 6, 2025

(54) HIGH-THROUGHPUT AND HIGH-PRECISION MEASUREMENT OF SINGLE-CELL DENSITY

(71) Applicant: Massachusetts Institute of Technology, Cambridge, CA (US)

(72) Inventors: Scott R. Manalis, Cambridge, MA (US); Weida Wu, Medford, MA (US); Teemu Petteri Miettinen, Cambridge, MA (US)

(73) Assignee: Massachusetts Institute of Technology, Cambridge, MA (US)

(21) Appl. No.: 18/715,053

(22) PCT Filed: Dec. 1, 2022
(86) PCT No.: PCT/US2022/051503

§ 371 Date: May 30, 2024

Related U.S. Application Data
(60) Provisional application No. 63/285,180, filed on Dec. 2, 2021.

(51) Int. Cl. G01N 9/24; G01N 15/10; G01N 15/14

(52) U.S. Cl. CPC G01N 9/24; G01N 15/1484; G01N 2015/1006

(57) ABSTRACT

Systems and methods for determining a property (e.g., density) of a particle (e.g., a cell) are generally described.

### RELATED APPLICATIONS

This application claims priority under 35 U.S.C. § 119 (e) to U.S. Provisional Patent Application No. 63/285,180, filed Dec. 2, 2021, and entitled “HIGH-THROUGHPUT AND HIGH-PRECISION MEASUREMENT OF SINGLE-CELL DENSITY,” which is incorporated herein by reference in its entirety for all purposes.

### TECHNICAL FIELD

Systems and methods for determining the density of particle, such as a cell, and related methods are generally described.

### BACKGROUND

The density of a cell is seldom measured because existing approaches for measuring the density of a cell with meaningful precision are both low throughput (e.g., a few hundred cells per afternoon) and complex. Accordingly, improved systems and methods are desired.

### SUMMARY

Systems and methods for determining a property (e.g., density) of a particle (e.g., a cell) are generally described. The subject matter of the present disclosure involves, in some cases, interrelated products, alternative solutions to a particular problem, and/or a plurality of different uses of one or more systems and/or articles.

In one aspect, a method for determining density of a particle is described, the method comprising flowing the particle through a microfluidic channel configured to receive the particle; driving the microfluidic channel with an excitation element; sensing a resonance frequency of the microfluidic channel as the particle flows through the microfluidic channel; exposing the particle to electromagnetic radiation and detecting an electromagnetic radiation signal; and determining the density of the particle based upon the resonance frequency and electromagnetic radiation signal.

In another aspect a method for determining density of a particle is described, the method comprising flowing a plurality of particles through a microfluidic channel configured to receive the particle; driving the microfluidic channel with an excitation element; sensing a plurality of resonance frequencies of the suspended microchannel resonator as each of the particles flows through the suspended microchannel resonator; exposing the plurality of particles to electromagnetic radiation and detecting an electromagnetic radiation signal for each of the particles; and determining the density of each of the particles based upon at least a portion of the plurality of resonance frequencies, wherein a coefficient of variation of an average of the densities is less than or equal to 1%.

In another aspect, a microfluidic system for determining a density of a particle is described, the system comprising a first microfluidic channel associated with an inlet; an excitation element for driving the first microfluidic channel; a second microfluidic channel in fluidic communication with the first microfluidic channel; a source of electromagnetic radiation associated with the second microfluidic channel; and a sensor associated with the suspended microchannel resonator configured to sense a resonance frequency of the suspended microchannel resonator; a detector configured to detect an electromagnetic radiation signal and associated with the second microfluidic channel; and a processor for determining the density of a particle based upon the electromagnetic radiation signal and resonance frequency of the microfluidic channel.

Other advantages and novel features of the present disclosure will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic illustration of a system for determining a property of a particle, the system comprising, at least, a microfluidic channel than can resonate and a source of electromagnetic radiation, according to some embodiments;

FIG. 2 is a plot depicting the fluorescent readout as a function of time for a plurality of cells as they flow within a microfluidic channel, according to some embodiments;

FIG. 3A is a schematic diagram a system comprising a suspended microchannel resonator and a fluorescence excitation region for determining the density of cells within a microfluidic system, according to some embodiments;

FIG. 3B is a plot depicting the fluorescent readout as a function of time for a plurality of cells as they flow within a microfluidic channel, according to some embodiments;

FIG. 3C shows the calculation of volume, buoyant mass, and density of a cell, according to some embodiments;

FIG. 4 is a plot of the determined volume of at least some of each cell within a plurality of cells as a function of the density of each cell, according to some embodiments;

FIG. 5A shows a set of plots fitting the densities of particles using a normal distribution fit and a alpha-stable distribution fit, according to some embodiments;

FIG. 5B is a plot of densities of particles vs. the Kolmogorov-Smirnov P value test to demonstrate that the alpha stable distribution provides a clear biomarker compared to normal and longnormal distributions, according to some embodiments;

FIG. 5C shows a set of plots in densities are fit with alpha and beta distributions for densities of populations of cells, according to some embodiments;

FIG. 6 is a series of micrographs showing posts imaged at different vertical adjustments of the microscope objective, according to some embodiments;

FIG. 7A is a plot of density measurements as a function of time for a plurality of cells as they flow through a microfluidic channel, according to some embodiments;

FIG. 7B is a plot showing the kernel density estimation as a function of density at different times when measuring a density of a plurality of cells flowing through a microfluidic channel, according to some embodiments;

FIG. 7C is a plot of density as a function of normalized volume exclusion PMT baseline of cells flowing through a microfluidic channel over the course of three replicates, according to some embodiments;

FIG. 7D is a series of micrographs depicting an air bubble within a microfluidic channel at the beginning of an experiment, at the end of the experiment, and after washing the microfluidic channel with bleach, according to some embodiments; and

FIG. 7E is a plot of density as a function of normalized volume exclusion PMT baseline of cells flowing through a microfluidic channel over the course of three replicates, wherein the microfluidic channel was washed with bleach between each replicate, according to some embodiments.

### DETAILED DESCRIPTION

Systems and methods for determining the density of a particle (e.g., a single cell) are generally described herein. Advantageously, determining the density of a single cell may be, in some cases, a desirable biophysical property as compared to mass or volume. For example, as described herein, the coefficient of variation (CV) of determining density in accordance with the embodiments described herein is generally smaller than the CV of determining mass or volume. Without wishing to be bound by theory, cells tightly regulate their density throughout the cell cycle even while the mass and/or volume of the cell may undergo significant changes. Therefore, using cell density as a biomarker may provide a useful method to detect a difference between cells at different biological conditions (e.g., cells at homeostasis vs. cells not at homeostasis, normal cells vs. cancerous cells). Despite this advantage, cell density is seldom measured because the existing techniques for doing so lack precision and are both low throughput and complex. And while various embodiments may include the determination of the CV, other data treatments are also possible as this disclosure is not so limited. Additional data treatments are described below.

The present disclosure describes systems and methods for determining the density of particle (e.g., a cell). These systems and methods may include a microfluidic channel (e.g., a microfluidic channel of a suspended microchannel resonator) that the particle may flow through and a source of electromagnetic radiation that can emit electromagnetic radiation (e.g., light) at the particle. The microfluidic channel may be or may comprise a suspended microchannel resonator (SMR), which can be used to determine the mass (e.g., the buoyant mass) of the particle, while the electromagnetic radiation can be used to determine the volume of the particle, and the density of the particle can then be determined from these two parameters. Advantageously, these systems and methods may determine the density of a cell in a straightforward manner with a relatively high throughput when compared to existing techniques that cannot precisely measure the density of cells.

In some embodiments, the systems and methods may be used to determine the density of a plurality of particles, such as one or more populations of cells. In some such embodiments, the density of the cells may provide other information about the cells (e.g., the biological condition of the cells), such that the cells can be sorted into different subpopulations based, at least in part, on the density of the cells. Advantageously, the systems and methods described herein can determine the density of a cell with high precision. For example, in some embodiments, the determined density has a resolution of 0.001 g/mL, which may be sufficient for resolving inherent biological variation of various cell types, which typically have a standard deviation in the range of 0.005 to 0.01 g/mL. Of course, it may also allow for the determination of the mass and/or the volume of each cell within a population of cells. And while many of the exemplary embodiments described herein describe the determination of cell density, it should be understood that the systems and methods described herein may also be suitable for determining other properties of particles (e.g., mass, volume).

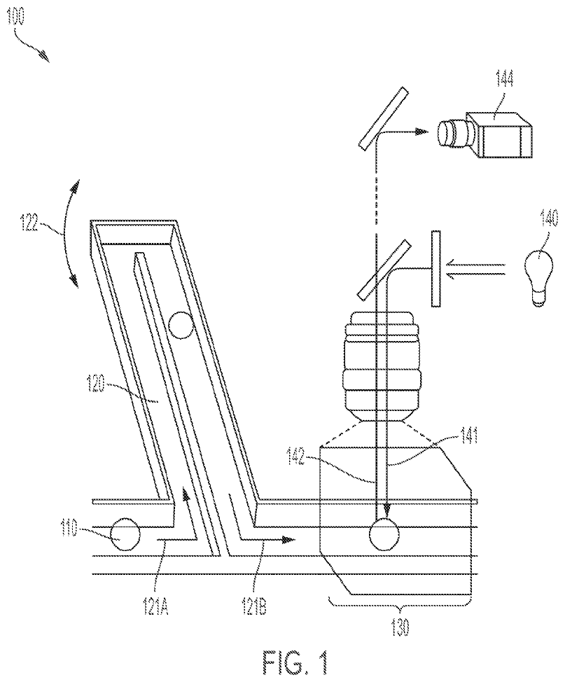

In some embodiments, the method includes flowing the particle through a microfluidic channel configured to receive the particle. For example, as illustrated schematically in FIG. 1, a system 100 comprises a particle 110 flowing within a microfluidic channel 120. Arrows 121A and 121B indicate the direction of flow of the particle (i.e., a fluid containing the particle). In some embodiments, the systems and methods include flowing a plurality of particles through a microfluidic channel configured to receive the particle. In some such embodiments, the system is configured to receive more than one particle (e.g., at least 2 particles, at least 10 particles, at least 102 particles, at least 103 particles, at least 104 particles, at least 105 particles, at least 106 particles, at least 107 particles, at least 108 particles) and the density (or some other property) of each cell can be determined.

In some embodiments, the method includes driving the microfluidic channel with an excitation element. For example, in FIG. 1, a driving element (not pictured) provides resonation to the microfluidic channel 120, shown schematically as resonation 122. As described in more detail elsewhere herein, resonating the microfluidic channel may be used to determine the mass (e.g., the buoyant mass) of a particle within the microfluidic channel.

In some embodiments, the method includes sensing a resonance frequency of the microfluidic channel as the particle flows through the microfluidic channel. In some embodiments, the measured resonant frequency is related to the mass, such as a buoyant mass, of the particle. In some embodiments, the method includes sensing a plurality of resonance frequencies of the suspended microchannel resonator as each of the particles flows through the suspended microchannel resonator.

In some embodiments, the method comprises exposing the particle to electromagnetic radiation and detecting an electromagnetic radiation signal. By way of illustration, FIG. 1 schematically depicts a light source 140 emitting light 141. In some embodiments, the particle may emit a signal 142, which can be detected by a detector 144. The signal detected by the detector may indicate a property of the particle, such as a particle volume. That is to say, in some embodiments, the signal is related to the volume of the particle.

In some embodiments, the method includes determining the density of the particle based, at least in part, upon the resonance frequency and electromagnetic radiation signal. In some such embodiments, the resonance frequency may provide information about the mass of the particle and the electromagnetic radiation signal may provide information about the volume of particle, and this information can be used to determine the density of the particle. In some embodiments, the density of each particle of a plurality of particles is determined. In some embodiments, the density of at least some (but not necessarily all) of the particles of the plurality of particles is determined.

The microfluidic systems described herein can be used for determining a density of a particle. In some embodiments, other properties of a particle (e.g., mass, volume) may also be determined. In some embodiments, the particle is a single cell. In some embodiments, the density of each cell of a plurality of cells is determined. In some such embodiments, each cell of the plurality of cells may be sorted based, at least in part, on the determining the density.

In some embodiments, a coefficient of variation of an average of the densities of the plurality of particles (e.g., sorted particles, unsorted particles) is less than or equal to 1%, less than or equal to 0.5%, less than or equal to 0.1%, less than or equal to 0.05%, less than or equal to 0.01%, or less than or equal to 0.005%. In some embodiments, the coefficient of variation of the average of the densities of the plurality of particles is greater than or equal to 0.001%, greater than or equal to 0.005%, greater than or equal to 0.01%, greater than or equal to 0.05%, greater than or equal to 0.1%, or greater than or equal to 0.5%. Combinations of the above-referenced ranges are also possible (e.g., less than or equal to 1% and greater than or equal to 0.001%). Other ranges are also possible. The coefficient of variation, as used herein, is given its ordinary meaning in the art and generally refers to a ratio of a sample standard deviation to a sample mean.

The particle may be any particle suitable to flow within the microfluidic channel. In some embodiments, the particle is a biological entity. Non-limiting examples of biological entities include virions, bacteria, protein complexes, exosomes, cells, or fungi (e.g., yeast). In some embodiments, the biological entity is obtained from a subject. A “subject” refers to any animal (e.g., a mammal, a human). Non-limiting examples of subjects include a human, a non-human primate, a cow, a horse, a pig, a sheep, a goat, a dog, a cat, a bird, a fish, or a rodent such as a mouse, a rat, a hamster, or a guinea pig. In an exemplary embodiment, the biological entity is a human cell. In some embodiments, the biological entity comprises a plant (e.g., algae, moss, ferns, shrubs, cactus, aloc, daffodils) and/or a plant-derived biological species such as plant cells. In some embodiments, the systems and methods described herein are useful for separating biological entities into a microfluidic channel from a plurality of biological entities obtained from a subject (e.g., a population of cells from a human), for example, determining one or more physical properties of the biological entity (e.g., density) and sorting on the basis of the one or more physical properties. In some such embodiments, the one or more physical properties may be used for diagnostic purposes.

The systems and methods described herein may be particularly useful for measuring physical properties (e.g., mechanical properties such as stiffness and/or Young's clastic modulus, a cross-linking density, a transport rate of small molecules into/out of the particle) of single particles such as individual cells (e.g., bacteria, yeast, liquid tumor cells, solid tumor cells suspended in fluid, immune cells). Such systems and methods may also be useful for measuring physical properties of a plurality of cells. As mentioned elsewhere herein, in some embodiments, such systems and methods may be used to determine the density (e.g., an average density) of a plurality of cells.

In some embodiments, the particle (e.g., a single particle, a plurality of particles, a cell, a plurality of cells) is provided in a fluid. As used herein, a “fluid” is given its ordinary meaning, i.e., a liquid or a gas. A fluid cannot maintain a defined shape and will flow during an observable time frame to fill a container or channel in which it is put. Thus, the fluid may have any suitable viscosity that permits flow. In some embodiments, the particles are in disordered arrangement in the fluid. In some cases, the particles are randomly distributed in the fluid. In some embodiments, the fluid is a liquid. In some embodiments, the fluid comprises water, a reagent, a solvent, a buffer, a cell-growth medium, or combinations thereof. In some embodiments, the particles are relatively soluble in the fluid.

Fluids can be introduced (e.g., transported, flowed, displaced) into the system (or a microfluidic channel therein) using any suitable component, for example, a pump, syringe, pressurized vessel, or any other source of pressure (e.g., a hydrostatic pressure gradient). Alternatively, fluids can be pulled into a microfluidic channel by the application of vacuum or reduced pressure on a downstream position (e.g., a vacuum port) of the system. Vacuum may be provided by any source capable of providing a lower pressure condition than exists upstream and/or downstream of a microfluidic channel of a system. Such sources may include vacuum pumps, aspirators, syringes, and evacuated containers. It should be understood, however, that in certain embodiments, methods described herein can be performed without a pressure drop across a microfluidic channel by using capillary flow, the use of valves, or other external controls that can vary pressure and/or flow rate of a microfluidic channel.

In some embodiments, a single particle (e.g., single cell) may be separated from a plurality of particles (e.g., a population of cells). In some embodiments, the amount of the particles used to separate a single particle from the plurality of particles is less than a total amount of particles in the plurality of particles. In some embodiments, the amount of particles in a fluid (e.g., a fluid flowing through a microfluidic channel) is greater than or equal to 100 particles per mL, greater than or equal to 250 particles per mL, greater than or equal to 500 particles per mL, greater than or equal to 1,000 particles per mL, greater than or equal to 2,500 particles per mL, or greater than or equal to 5,000 particles per mL of fluid. Advantageously, a single particle may be separated from a plurality of particles (e.g., plurality of biological entities) having a relatively large amount of particles per unit volume, using the method and systems described herein. For example, in some embodiments, the amount of particles in the fluid is greater than or equal to 10,000 particles per mL, greater than or equal to 25,000 particles per mL, greater than or equal to 50,000 particles per mL, greater than or equal to 100,000 particles per mL, greater than or equal to 150,000 particles per mL, greater than or equal to 200,000 particles per mL, greater than or equal to 250,000 particles per mL, greater than or equal to 300,000 particles per mL, greater than or equal to 350,000 particles per mL, greater than or equal to 400,000 particles per mL, greater than or equal to 450,000 particles per mL, greater than or equal to 500,000 particles per mL, greater than or equal to 550,000 particles per mL, greater than or equal to 600,000 particles per mL, greater than or equal to 650,000 particles per mL, greater than or equal to 700,000 particles per mL, greater than or equal to 750,000 particles per mL, greater than or equal to 800,000 particles per mL, greater than or equal to 850,000 particles per mL, greater than or equal to 900,000 particles per mL, or greater than or equal to 950,000 particles per mL of fluid. In some embodiments, the amount of particles in the fluid is less than or equal to 1,000,000 particles per mL, less than or equal to 950,000 particles per mL, less than or equal to 900,000 particles per mL, less than or equal to 850,000 particles per mL, less than or equal to 800,000 particles per mL, less than or equal to 750,000 particles per mL, less than or equal to 700,000 particles per mL, less than or equal to 650,000 particles per mL, less than or equal to 600,000 particles per mL, less than or equal to 550,000 particles per mL, less than or equal to 500,000 particles per mL, less than or equal to 450,000 particles per mL, less than or equal to 400,000 particles per mL, less than or equal to 350,000 particles per mL, less than or equal to 300.000 particles per mL, less than or equal to 250,000 particles per mL, less than or equal to 200,000 particles per mL, less than or equal to 150,000 particles per mL, less than or equal to 100,000 particles per mL, less than or equal to 50,000 particles per mL, or less than or equal to 25,000 particles per mL of fluid. In some embodiments, the amount of particles in the fluid is less than or equal to 7,500 particles per mL, less than or equal to 5,000 particles per mL, less than or equal to 2,500 particles per mL, less than or equal to 1,000 particles per mL, less than or equal to 500 particles per mL, or less than or equal to 250 particles per mL. Combinations of the above-referenced ranges are also possible (e.g., greater than or equal to 100 particles per mL and less than or equal to 10,000 particles per mL, greater than or equal to 100 particles per mL and less than or equal to 1,000,000 particles per mL, greater than or equal to 10,000 particles per mL and less than or equal to 1,000,000 particles per mL, greater than or equal to 10,000 particles per mL and less than or equal to 750,000 particles per mL, greater than or equal to 500,000 particles per mL and less than or equal to 750,000 particles per mL). Other ranges are also possible.

In some embodiments, a property of a particle (e.g., a single particle) in a plurality of particles may be determined at a variety of rates. In some embodiments, a property of a particle in a plurality of particles is determined at a rate of greater than or equal to 500 particles per hour, greater than or equal to 750 particles per hour, greater than or equal to 1000 particles per hour, greater than or equal to 1500 particles per hour, greater than or equal to 2000 particles per hour, greater than or equal to 2500 particles per hour, greater than or equal to 3000 particles per hour, greater than or equal to 4000 particles per hour, greater than or equal to 5000 particles per hour, or greater than or equal to 7500 particles per hour. In some embodiments, the property of the plurality of particles is determined at a rate of less than or equal to 7500 particles per hour, less than 5000 particles per hour, less than or equal to 4000 particles per hour, less than or equal to 3000 particles per hour, less than or equal to 2500 particles per hour, less than or equal to 2000 particles per hour, less than or equal to 1500 particles per hour, less than or equal to 1000 particles per hour, less than or equal to 750 particles per hour, or less than or equal to 500 particles per hour. Combinations of the above-referenced ranges are also possible (e.g., greater than or equal to 750 particles per hour and less than or equal to 7500 particles per hour). Other ranges are possible.

In an exemplary embodiment, the fluid comprises a plurality of cells, and the amount of cells within the fluid is greater than or equal to 10,000 particles per mL and less than or equal to 750,000 particles per mL. Advantageously, the systems and methods described herein may enable the loading of separated particle(s) into a channel at a particular frequency (e.g., less than or equal to 1 particle per 10 seconds) and/or spacing (e.g., greater than or equal to 1 mm) irrespective of the amount of particles in the fluid, as compared to passive loading of cells into channels. That is to say, in some embodiments, substantially the same method and/or system may be used to separate particles at a particular frequency and/or spacing within a microfluidic channel, without diluting (or concentrating) the particles in the fluid prior to loading the particle-containing fluid into the system. In some embodiments, a relatively high amount of particles in the fluid are in a disordered arrangement.

In some embodiments, the system (e.g., the microfluidic system) comprises one or more microfluidic channels. “Microfluidic channels” generally refer to channels having an average cross-sectional dimension of less than or equal to 1 mm. In some embodiments, the one or more microfluidic channels may be open (e.g., not fully enclosed, partially open to air). In some embodiments, the one or more microfluidic channels may be partially or entirely enclosed. The one or more microfluidic channel can be any suitable channel, conduit, passage, or the like, and those skilled in the art in view of this disclosure will be capable of selecting appropriate channel. In some instances, the channel may be associated with (e.g., in fluidic communication with) an inlet and/or an outlet, in addition to one or more other channels. In some embodiments, the microfluidic channel is or includes a suspended microchannel resonator (SMR), which may be configured to resonate (e.g., during or after being driven by a driving element). In some embodiments, the microfluidic system comprises a first microfluidic channel associated with an inlet. In some embodiments, the microfluidic system comprises a second microfluidic channel in fluidic communication with the first microfluidic channel. In some embodiments, a plurality of microfluidic channels is present in a system and may be operated in tandem, such that the system may determine the property of several particles (or more) in tandem, each using a microfluidic channel to determine the property. In some embodiments, a single microfluidic channel is used, wherein a single property of several particles (or more) is measured. In some cases, a single microfluidic channel is used wherein multiple properties (e.g., at least 2 properties) of several particles (or more) may be measured serially (e.g., a first property measured at a first location within the single microfluidic channel, a second property measured at a second location within the single microfluidic channel, and so forth).

The microfluidic channels (e.g., a first microfluidic channel, a second microfluidic channel) of the system may have a particular average cross-sectional dimension. The “cross-sectional dimension” (e.g., a width, a height, a radius) of the channel is measured perpendicular to the direction of fluid flow. In some embodiments, the average cross-sectional dimension of a microfluidic channel is less than or equal to 1 mm, less than or equal to 800 μm, less than or equal to 600 μm, less than or equal to 500 μm, less than or equal to 400 μm, less than or equal to 300 μm, less than or equal to 200 μm, less than or equal to 100 μm, less than or equal to 50 μm, less than or equal to 25 μm, less than or equal to 20 μm, less than or equal to 15 μm, or less than or equal to 10 μm. In some embodiments, the average cross-sectional dimension of a microfluidic channel is greater than or equal to 5 μm, greater than or equal to 10 μm, greater than or equal to 15 μm, greater than or equal to 20 μm, greater than or equal to 25 μm, greater than or equal to 50 μm, greater than or equal to 100 μm, greater than or equal to 200 μm, greater than or equal to 300 μm, greater than or equal to 400 μm, greater than or equal to 500 μm, greater than or equal to 600 μm, or greater than or equal to 800 μm. Combinations of the above-referenced ranges are also possible (e.g., greater than or equal to 5 μm and less than or equal to 1 mm). Other ranges are also possible.

In some embodiments, a microfluidic channel of the system (e.g., the first microfluidic channel, the second microfluidic channel) may have any suitable cross-sectional shape (e.g., circular, oval, triangular, irregular, trapezoidal, square or rectangular, serpentine, u-shaped, or the like). A microfluidic channel may also have an aspect ratio (length to average cross-sectional dimension) of at least 2:1, more typically at least 3:1, 5:1, or 10:1 or more. A fluid within the microfluidic channel may partially or completely fill the fluidic channel.

In some embodiments, particles flow in the microfluidic channel at a particular average velocity along the longitudinal axis of the microfluidic channel. In some embodiments, the average velocity of particles along the longitudinal axis of the microfluidic channel is greater than or equal to 0.05 mm/second, greater than or equal to 0.1 mm/second, greater than or equal to 0.25 mm/second, greater than or equal to 0.5 mm/second, greater than or equal to 0.75 mm/second, greater than or equal to 1 mm/second, greater than or equal to 2 mm/second, greater than or equal to 3 mm/second, greater than or equal to 4 mm/second, greater than or equal to 5 mm/second, greater than or equal to 6 mm/second, greater than or equal to 7 mm/second, greater than or equal to 8 mm/second, greater than or equal to 9 mm/second, greater than or equal to 10 mm/second, greater than or equal to 20 mm/second, greater than or equal to 30 mm/second, greater than or equal to 40 mm/second, greater than or equal to 50 mm/second, greater than or equal to 60 mm/second, greater than or equal to 70 mm/second, greater than or equal to 80 mm/second, or greater than or equal to 90 mm/second. In some embodiments, the average velocity of particles along the longitudinal axis of a microfluidic channel is less than or equal to 100 mm/second, less than or equal to 90 mm/second, less than or equal to 80 mm/second, less than or equal to 70 mm/second, less than or equal to 60 mm/second, less than or equal to 50 mm/second, less than or equal to 40 mm/second, less than or equal to 30 mm/second, less than or equal to 20 mm/second, less than or equal to 10 mm/second, less than or equal to 9 mm/second, less than or equal to 8 mm/second, less than or equal to 7 mm/second, less than or equal to 6 mm/second, less than or equal to 5 mm/second, less than or equal to 4 mm/second, less than or equal to 3 mm/second, less than or equal to 2 mm/second, less than or equal to 1 mm/second, less than or equal to 0.75 mm/second, less than or equal to 0.5 mm/second, or less than or equal to 0.25 mm/second. Combinations of the above-referenced ranges are also possible (e.g., greater than or equal to 0.05 mm/second and less than or equal to 100 mm/second). Other ranges are also possible.

In some embodiments, a microfluidic channel (e.g., a first microfluidic channel, a second microfluidic channel) may have a variety of suitable configurations. In some embodiments, at least a portion of the microfluidic channel may be substantially linear in the direction of fluid flow. In some embodiments, at least a portion of the microfluidic channel may be curved, bent, serpentine, staggered, zigzag, spiral, or combinations thereof. Advantageously, the use of a non-linear fluidic channels may permit the incorporation of two or more microfluidic channels into the system (e.g., such that a plurality of particles may be measured in parallel, such that a change in a property of a single particle may be determined, e.g., in a plurality of microfluidic channels, either in series or in parallel or some combination of both).

The systems and methods described herein may also include an excitation element. The excitation element may drive or cause resonation in a microfluidic channel (e.g., a first microfluidic channel). In some embodiments, the system comprises one or more components for oscillating a microfluidic channel and/or measuring the oscillation (and/or resonant frequency) of the microfluidic channels. For example, in some embodiments, the system comprises an actuator configured to vibrate (e.g., oscillate) the microfluidic channel (e.g., at a particular frequency and/or bending mode). In some embodiments, the actuator is configured to vibrate the microfluidic channel comprising a suspended microchannel resonator. In some cases, as a particle (e.g., a biological entity, a cell) flows through the suspended microchannel resonator, the mass of the particle may cause a change in the frequency at which the suspended microchannel resonator resonates (e.g., the resonance frequency changes). While many of the embodiments described herein generally refer to a ‘microfluidic channel’, those of ordinary skill in the art would understand based upon the teachings of this specification that, in some embodiments, the microfluidic channel may be a channel of a suspended microchannel resonator.

A source of electromagnetic radiation may also be present within the system. In some embodiments, the source of electromagnetic radiation is associated with a microfluidic channel (e.g., a second microfluidic channel). In some embodiments, the source of electromagnetic radiation may provide light to a particle (e.g., a single cell, a plurality of cells). In some such embodiments, the provided light may trigger a luminescence event (e.g., fluorescence) within or associated with the particle, which may act as a signal. In some such embodiments, the signal may provide information about the volume of the particle. In some embodiments, the electromagnetic radiation signal is a luminescence signal within the particle. In some embodiments, the electromagnetic radiation signal comprises a difference between electromagnetic radiation detected in the presence of the particle and electromagnetic radiation detected in the absence of the particle under substantially identical conditions. In some cases, the electromagnetic radiation may illuminate a portion of the microfluidic channel, wherein the fluid in the microfluidic channel comprises a fluorophore. In some such cases, in the absence of a particle, the fluorophore fluoresces, and a first electromagnetic radiation signal is measured. In other such cases, in the presence of a particle, the fluorophore fluoresces, and a second electromagnetic radiation signal is measured, wherein the second electromagnetic radiation signal is less than the first electromagnetic radiation signal due to the presence of the particle (e.g., because the particle does not fluoresce).

In some embodiments, the electromagnetic radiation signal may vary over time. In some such cases, air bubbles accumulating within the microfluidic channel while in use and may contribute to the variation observed in the electromagnetic radiation signal. In some such cases, to remove air bubbles and to mitigate any associated variation in the electromagnetic radiation signal, the microfluidic channel may be washed with a solution (e.g., an aqueous solution). In some such embodiments, the solution comprises bleach (e.g., a solution of sodium hypochlorite). In this manner, in some embodiments, measurements obtained (e.g., volume, density) using the methods disclosed elsewhere herein maintain relatively high precision when compared to measurements made using conventional approaches.

In some embodiments, the volume of a particle (e.g., a cell) may be determined, for example, by weighing the particle in the suspended microchannel using two fluids with different densities. In some embodiments, the node deviation is normalized by a median volume (e.g., a median volume of a plurality of particles of the same type). Suitable methods for determining the volume of a single particle (or plurality of particles) are generally described in commonly-owned U.S. Pat. No. 8,087,284, entitled “Method And Apparatus For Measuring Particle Characteristics Through Mass Detection”, issued Jan. 3, 2012, which is incorporated herein by reference in its entirety for all purposes.

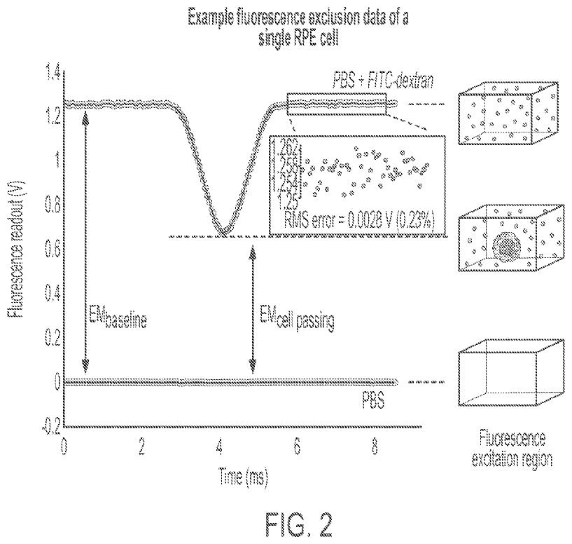

In some embodiments, the volume of a particle may be determined using volume exclusion (e.g., prior to buoyant mass measurement). For example, as illustrated in FIG. 2, electromagnetic radiation may be used to determine the volume of the particle. In some embodiments, the volume of the cell, is determined by measuring the electromagnetic radiation signal (e.g., fluorescence intensity) of a channel (relative to background) in the presence and absence of the particle. For example, in some embodiments, the volume of a particle (Vₚ) is:

$${V}_{p} = ( {EM}_{baseline} - {EM}_{p} ) / ( {EM}_{baseline} ) * {V}_{o} ,$$

where EM_{baseline} is the electromagnetic radiation signal in the channel (relative to background) in the absence of the particle and EMₒ is the electromagnetic radiation signal of the channel (relative to background) in the presence of the particle, and Vₒ is the calibration volume.

In some embodiments, the density of the particle may be determined by calculating a ratio of the buoyant mass of the particle (e.g., as determined by the suspended microchannel resonator) and the volume of the particle.

In some embodiments, a sensor is associated with a microfluidic channel (e.g., a first microfluidic channel, a suspended microchannel resonator). In some cases, the sensor may be configured to sense a resonance frequency of the suspended microchannel resonator. In some cases, the sensor is configured to sense when the resonance frequency of the suspended microchannel resonator changes. In some embodiments, the sensor senses a first resonance frequency when fluid (e.g., an aqueous solution) flows through the suspended microchannel resonator in the absence particles of interest (e.g., a biological entity, a cell) and senses a second resonance frequency when a particle of interest is present in the fluid in the suspended microchannel resonator, wherein the first resonance frequency and the second resonance frequency are different. In some such embodiments, the difference between the first resonance frequency and the second resonance frequency may be used to determine a parameter (e.g., mass, buoyant mass) of the particle of interest flowing through the suspended microchannel resonator.

In some embodiments, the system may also comprise a detector. In some cases, the detector is configured to detect electromagnetic radiation from the particle (e.g., a signal from the particle). In some embodiments, the detector is configured to detect electromagnetic radiation from a solution in which the particle is suspended. In some cases, the detector is configured to detect electromagnetic radiation from the solution in the presence and/or the absence of the particle. In some such cases, the difference in the electromagnetic radiation from the solution in the presence and the absence of the particle is an electromagnetic radiation signal. In some embodiments, the detector is configured to detect an electromagnetic radiation signal and is associated with the second microfluidic channel.

In some embodiments, a detected electromagnetic radiation signal is dependent on a distance between a microfluidic system (e.g., a microfluidic channel wherein a fluid comprising a particle of interest flows) and a detector. In some embodiments, the microfluidic system comprises posts, wherein the posts comprise a planar surface substantially parallel to a microscope stage on which the microfluidic system is placed. In some such cases, a microscope objective may focus on the planar surface of the posts and, in combination with a sensor, provide real-time feedback to the microfluidic system to maintain a distance between the microscope objective and the posts. Accordingly, in some such embodiments, the microfluidic system has relatively high mechanical stability (e.g., the amount that the distance between the microscope objective and the microfluidic system changes), wherein the plane of focus of the microscope objective remains less than or equal to 5 μm, less than or equal to 3 μm, or less than or equal to 1 μm from the plane of the microfluidic system. In turn, in some such embodiments, the distance between the detector and the microfluidic system also has the relatively high mechanical stability.

In some embodiments, the system also comprises a processor for determining the density of a particle based upon the electromagnetic radiation signal and resonance frequency of the microfluidic channel. In some such embodiments, the processor is a controller and/or microprocessor. In certain embodiments, the controller is configured (e.g., programmed) to receive and transmit data commands to/from one or more components of the system (e.g., a driving element, a sensor and/or detector). In some such embodiments, the data includes one or more signals from one or more detectors. In some such embodiments the controller and/or microprocessor is configured to determine the resonant frequency of a microfluidic channel (e.g., a suspended microchannel). In such some embodiments, the controller may be configured to adjust various parameters based on external metrics. For example, in certain embodiments, the controller is configured to adjust the oscillation frequency of a microfluidic channel in response to a signal from a user and/or a detector in electrical communication with the controller. In some such embodiments, the controller adjusts the oscillation frequency in response to a signal from the detector due to a particle in the microfluidic channel.

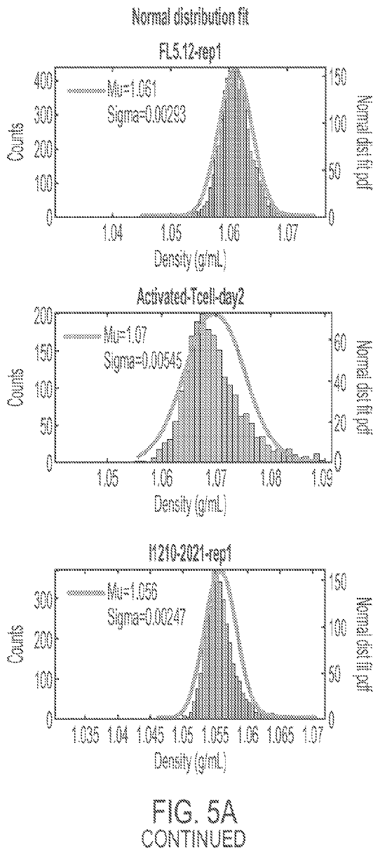

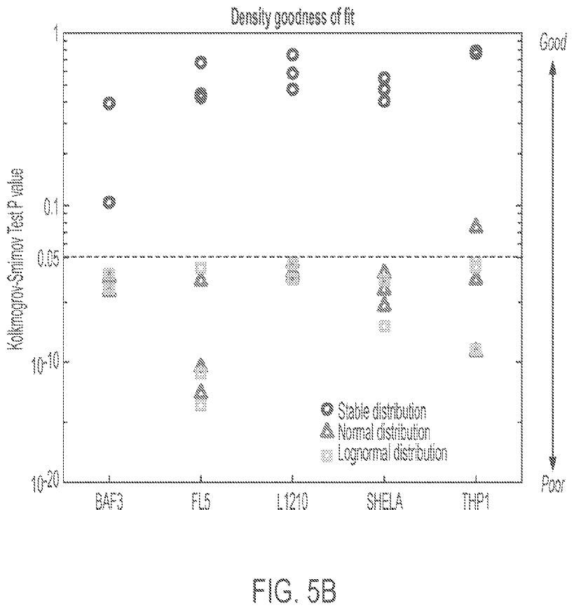

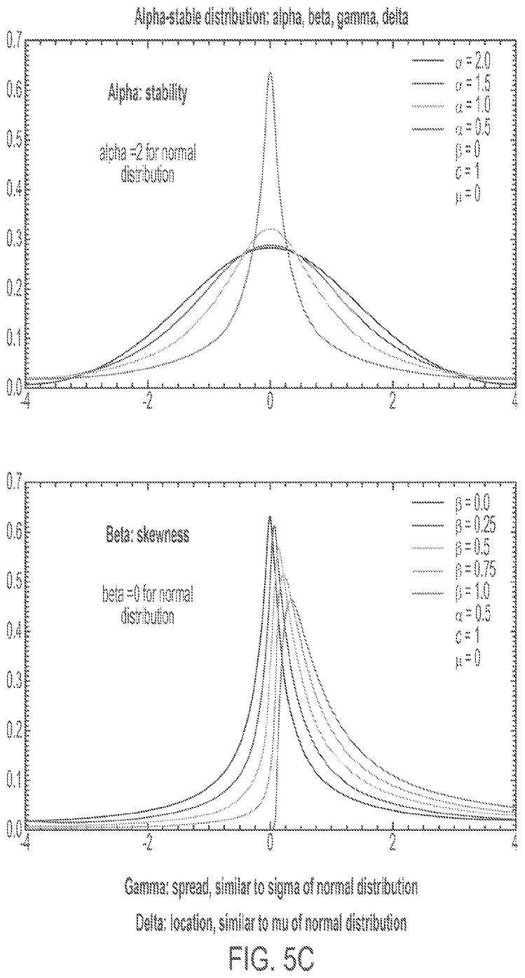

As mentioned above, in some embodiments, mass, volume, and/or density (e.g., an average mass, an average volume, an average density) may be determined for particles (e.g., cells). In some such embodiments, determination of mass, volume, and/or density may be performed in particular manner to provide a higher quality data treatment relative to conventional data treatments. For example, in some embodiments, a distribution of densities from a cell population can be described by an alpha-stable distribution fitting rather than a normal distribution fitting. By way of illustration (and not limitation), FIG. 5A schematically illustrates a normal distribution relative to an alpha-stable distribution. As can be seen in the figure, the fit for the alpha-stable distribution envelopes the data more closely relative to the normal distribution fit. This benefit is also observed in the plot in FIG. 5B. In some cases, there are (at least) four parameters that may be useful for determining a property (e.g., density) of a population of particles, including, but not limited to, shape, symmetry, spread, and/or location of the data. FIG. 5C illustrates an example of an alpha fit, and also a beta fit. In some cases, these parameters from an alpha-stable distribution fit may be useful biomarkers both individually, or in combination of each other. Of course, other statistical fits and/or regression models are possible as this disclosure is not so limited.

In some embodiments, a processor and/or a controller may include one or more proportional, integral (PI), and/or derivative (PID) feedforward and/or feedback loops to adjust the oscillation frequency of a microfluidic channel. The controller may be implemented by any suitable type of analog and/or digital circuitry. In one set of embodiments, the processor and/or the controller may be implemented in a field programmable gate array (FPGA). For example, the processor and/or the controller may be implemented using hardware or a combination of hardware and software. When implemented using software, suitable software code can be executed on any suitable processor (e.g., a microprocessor, FPGA) or collection of processors. The processor and/or the controller can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, or other tangible, non-transitory computer-readable storage medium) encoded with a computer program (i.e., a plurality of executable instructions) that, when executed on one or more processors, performs the above discussed functions of one or more embodiments. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The article(s), system(s), and device(s) or portions thereof (e.g., a microfluidic channel, a suspended microchannel resonator) described herein can be fabricated of any suitable material. Non-limiting examples of materials include polymers (e.g., polypropylene, polyethylene, polystyrene, poly(acrylonitrile, butadiene, styrene), poly(styrene-co-acrylate), poly(methyl methacrylate), polycarbonate, polyester, poly(dimethylsiloxane), PVC, PTFE, PET, or blends of two or more such polymers), adhesives, and/or metals including nickel, copper, stainless steel, bulk metallic glass, or other metals or alloys, or ceramics including glass, quartz, silica, alumina, zirconia, tungsten carbide, silicon carbide, or non-metallic materials such as graphite, silicon, or others.

The following applications are incorporated herein by reference, in their entirety, for all purposes: U.S. Publication No. US 2018/0299362, published on Oct. 18, 2018, filed as U.S. application Ser. No. 15/940,001 on Mar. 29, 2018, and entitled “SYSTEMS, ARTICLES, AND METHODS FOR FLOWING PARTICLES”; U.S. Publication No. US 2018/0245972, published on Aug. 30, 2018, filed as International Patent Application No. PCT/US2015/057634 on Oct. 27, 2015, and entitled “SIMULTANEOUS OSCILLATION AND FREQUENCY TRACKING OF MULTIPLE RESONANCES VIA DIGITALLY IMPLEMENTED PHASE-LOCKED LOOP ARRAY”; and U.S. Publication No. US 2021/0046477, published on Feb. 18, 2021, filed as U.S. application Ser. No. 16/901,924 on Jun. 15, 2020, and entitled “RAPID AND HIGH-PRECISION SIZING OF SINGLE PARTICLES USING PARALLEL SUSPENDED MICROCHANNEL RESONATOR ARRAYS AND DECONVOLUTION”.

The following examples are intended to illustrate certain embodiments of the present invention, but do not exemplify the full scope of the invention.

### Example 1

The following example describes the determination of single-cell densities from a population of cells using a suspended microchannel resonator (SMR) and a source of electromagnetic radiation source to determine the mass and the volume of the cells, respectively.

Single-cell density is a desirable biophysical property when compared to mass or volume because its CV (i.e., coefficient of variation) is more than 10-fold smaller than that of mass and/or volume. This is the case because cells tightly regulate their density throughout the cell cycle even though mass and volume undergo significant changes.

Despite these benefits in measuring the density of cell, it is seldom measured because the approach for measuring density of a cell with meaningful precision is both low throughput (e.g., a few hundred cells per afternoon) and complex (e.g., only advanced SMR users may be capable of precisely measuring). Despite the limitations of this approach, it is extremely precise, with a density resolution of 0.001 g/mL, which is sufficient for resolving the inherent biological variation of various cell types (which typically have a standard deviation in the range of 0.005 to 0.01 g/mL). Additionally, it may enable for the mass, density, and volume to be measured from each cell.

This example describes a new approach where volume exclusion is used to measure cell volume on the SMR chip immediately prior to the buoyant mass measurement. The principle of volume exclusion is shown schematically in FIG. 2.

Assuming linearity exists between the PMT (photomultiplier tube) voltage readout and the number of fluorophores being excited in the excitation region, then:

$$EM = λ \times {c}_{f} \times {V}_{excited} ,$$

where EM is the PMT readout in units of volts, λ is the linearity constant, c_{f} is the concentration of fluorophores, and V_{excited} is the volume of the excited fluorophore detected by the PMT.

Assuming the concentration of fluorophores in the flow chamber is uniform throughout a measurement, then:

$$κ = λ \times {c}_{f}$$

$$EM = κ \times {V}_{excited} ,$$

where κ is the linearity constant between EM and V_{excited}.

Thus, assuming complete exclusion of the fluorophore by the cell membrane and that the cell does not comprise any components that fluorescence at the same wavelength as the fluorophores:

$${EM}_{baseline} = κ \times {V}_{ER}$$

$${EM}_{cell passing} = κ \times ( {V}_{ER} - {V}_{cell} )$$

where EM_{baseline} is the PMT readout when no cell is passing, EM_{cell passing} is the PMT readout when a cell is passing and fully included in the excitation region, V_{ER} is the volume of the fluorescence excitation region, and V_{cell} is the cell volume.

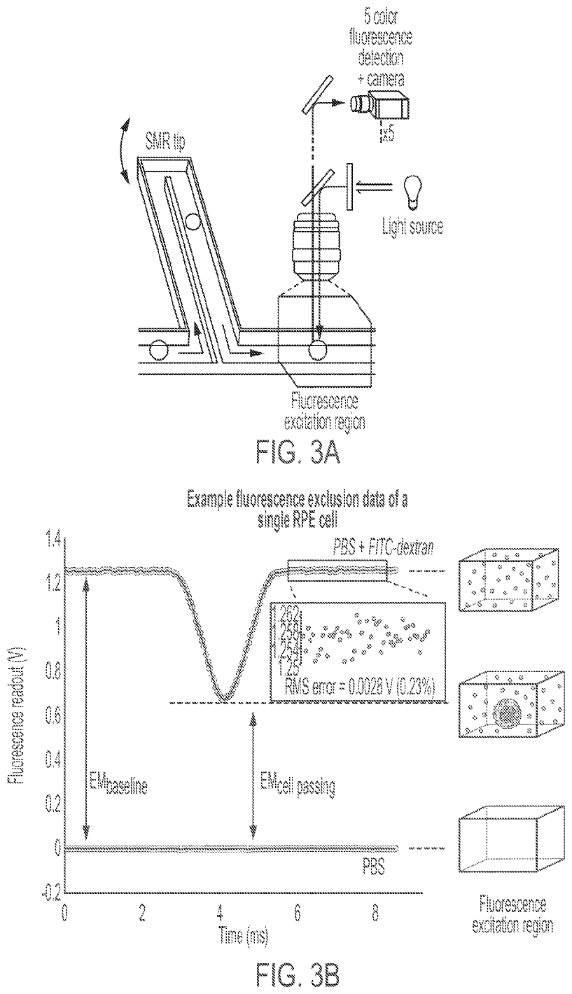

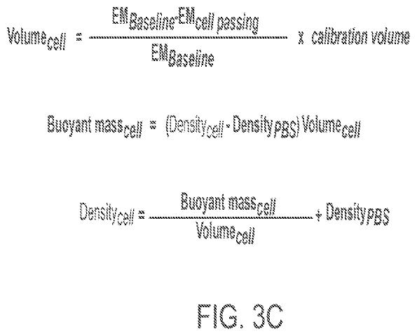

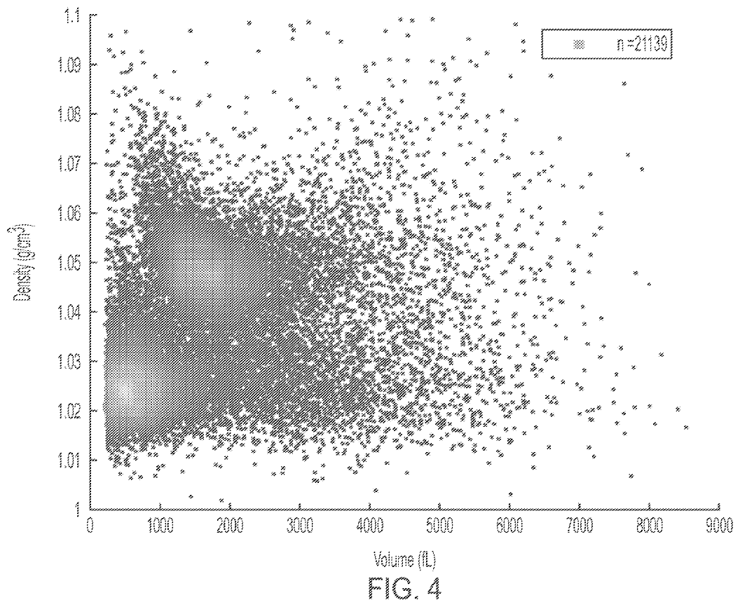

While volume exclusion has been known in some conventional systems, the integration of volume exclusion with the SMR buoyant mass (as shown schematically in FIG. 3) enables mass, volume, and density of each cell to be measured with a precision that reveals the inherent biological variation of density.

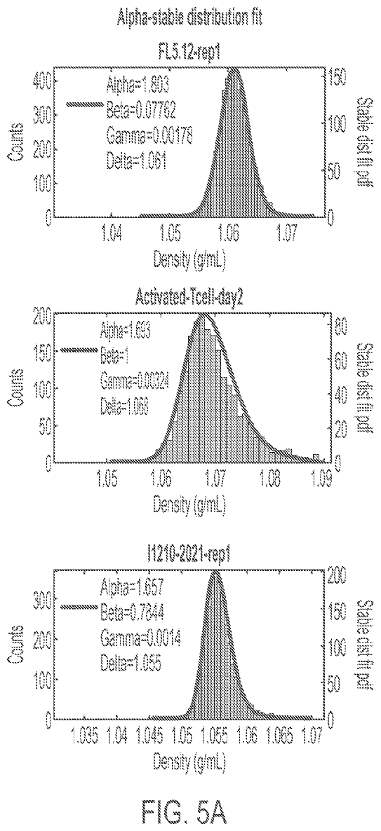

The approach may be similar to the measurement of buoyant mass (since fluid exchange is not required) and the throughput can approach tens of thousands of cells per hour (e.g., much greater than a few hundred cells per afternoon). An example of measurements from human tumor cells from a patient derived xenograft (PDX) is shown in FIG. 4.

### Example 2

The following example describes a method for increasing optical signal quality during experiments.

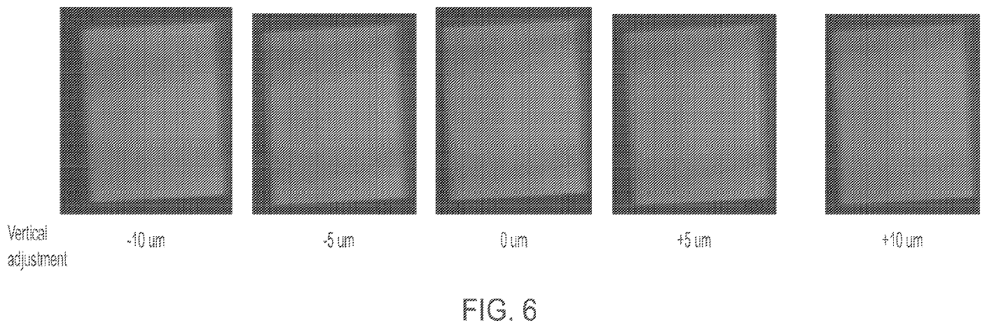

Experiments performed using microfluidic chips are highly sensitive to mechanical stability between the microscope and the microfluidic system. That is, if the distance changes between the microscope objective and the microfluidic system, the microscope objective is no longer focused on the plane of interest (e.g., where a microfluidic channel comprising a flowing fluid comprising particles of interest is located). Accordingly, posts of a fixed height were added between the microscope and the breadboard. For example, FIG. 6 shows a series of micrographs of the microfluidic channel taken from a camera connected to the microscope of the posts at different vertical adjustments of the microscope objective (e.g., the objective focused at −10 μm, −5 μm, 0 μm, +5 μm, and +10 μm from the plane of the top of the microfluidic channel). By further integrating a distance sensor that monitors the distance between the microscope objective and the plane of the posts while imaging and programming the distance sensor to provide real-time feedback to the system, the vertical distance between the microscope objective and the plane for the posts was controlled to within +1 μm from focus (e.g., +1 μm from 0 μm of the posts in FIG. 6).

### Example 3

The following example describes an approach for avoiding air bubbles that form along the microfluidic channel walls.

Signal integrity of serial measurements (e.g., multiple volume measurements, multiple density measurements) is dependent on each measurement (e.g., measurements at different times) occurring under similar or identical conditions. In some cases, air bubbles form along the microchannel walls when flushing devices with media comprising serum. In some such cases, air bubbles bias total illumination volume (e.g., by decreasing solution volume in the illumination pathway), thus creasing bias for the volume signal. Likewise, density measurements are also biased in such cases. For many existing systems, air bubbles can significantly bias experimental measurements (e.g., volume, density). This is because the observed shift in volume signals over time is relatively small and could be attributed to the heterogeneity of cell size in a biological sample. Moreover, the relatively low signal-to-noise ratio during volume measurements may prevent confirmation of experimental bias. By removing air bubbles in this manner, more precise volume measurements can be obtained, and may also improve resulting density measurements.

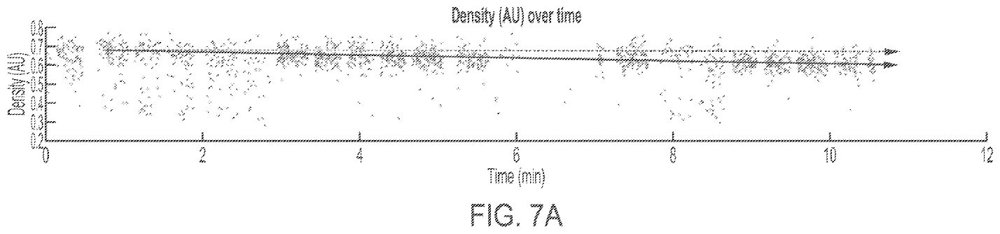

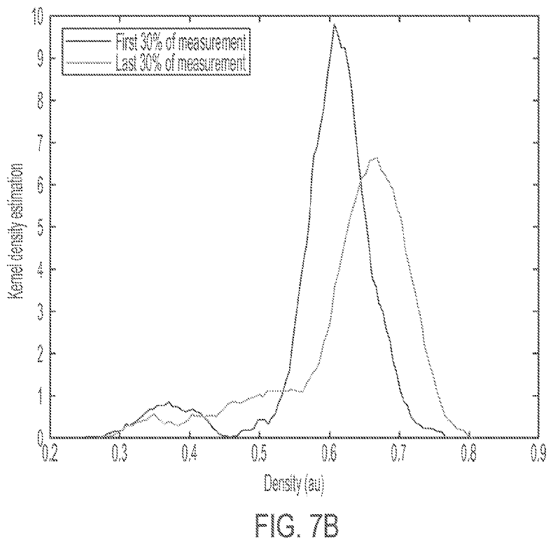

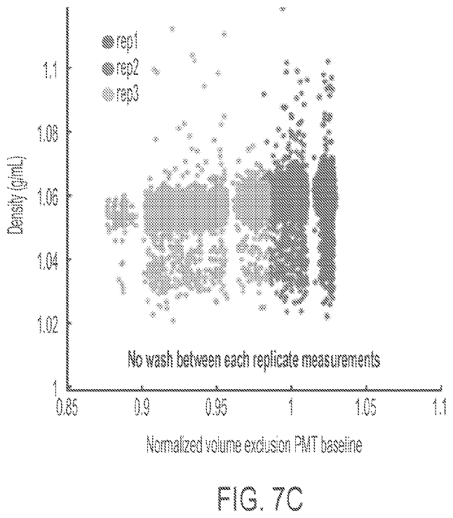

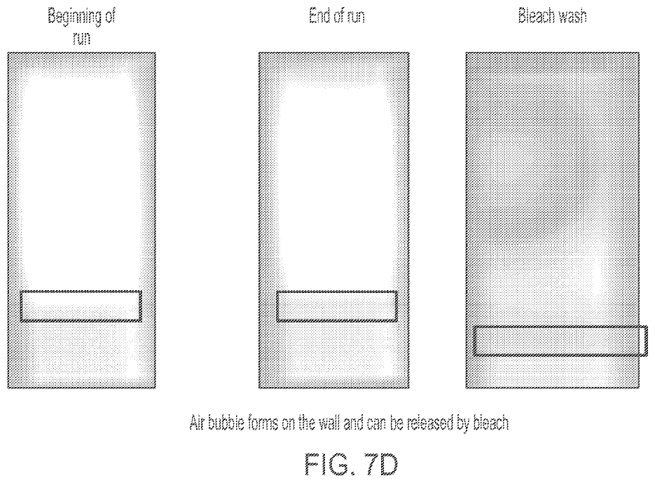

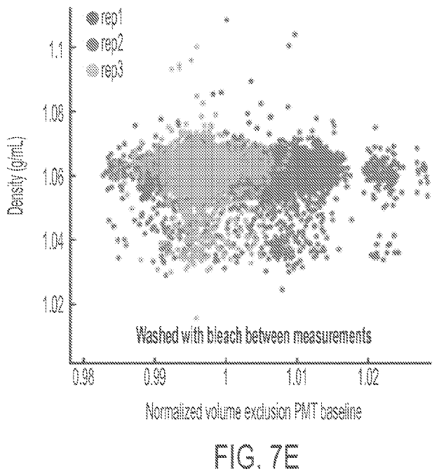

For example, FIG. 7A shows a plot of density measured as a function of time of cells flowing through a microfluidic system. Here, the measured data trend downwards as a function of time (solid, bottom arrow), indicating bias in density measurements away from ideal, unbiased conditions (dotted, top arrow).

Another example of biased measurements is shown in FIG. 7B, wherein a density estimate is plotted vs. the measured density at the beginning (e.g., first 30% of the measurement) and at the end (e.g., last 30% of the measurement) of cells flowing through a microfluidic system throughout an experiment. Here, the difference in the measured cell density from the beginning to the end of the experiment show differences (e.g., bias) arise over the course of the measurement.

Moreover, FIG. 7C is another exemplary plot showing biased measurements over the course of an experiment. Here, the measured density vs. the normalized volume exclusion PMT baseline is plotted over the course of three sequential replicates of cells flowing through a microfluidic system. In this exemplary embodiment, the device was not washed between the experimental replicates. The positive slope observed in the plot was likely caused by air bubble bias. Likewise, the biases observed in experiment measurements depicted in FIGS. 7A-7B were also attributed to the formation of air bubbles.

Identifying and removing measurement biases from methods is useful for obtaining relatively high precision between measurements. Accordingly, between experimental replicates, devices were washed with bleach to remove a source of bias (e.g., air bubbles accumulating in a microfluidic channel). FIG. 7B is a series of micrographs illustrating the results of the washing process. Here, at the beginning of the experimental run (e.g., measuring a property of each particle in a plurality of particles; first micrograph), the air bubble is negligible (box 1). At the end of the run (middle micrograph), the air bubble is much more prevalent (box 2, more dark shading than in box 1) than at the beginning of the run. To mitigate the accumulation the air bubble, the device was washed with bleach after the run (third micrograph), minimizing the air bubble (box 3), as shown schematically in FIG. 7D. The improvements derived from washing the device with bleach are plotted in FIG. 7E, wherein the measured density vs. the normalized volume exclusion PMT baseline is plotted over the course of three sequential replicates on the same device with bleach washing steps between replicate measurements. Here, the values obtained from replicates 1, 2, and 3 are more precise and are present with little-to-no bias (e.g., the data are not segregated by replicate and there is no slope in the measurement as a function of replicate) when compared to the previous results without the bleach washing step, as shown in FIG. 7C.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or.” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B.” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of.” “only one of.” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for determining a density of a particle, the method comprising:
flowing the particle through a microfluidic channel configured to receive the particle;
driving the microfluidic channel with an excitation element;
sensing a resonance frequency of the microfluidic channel as the particle flows through the microfluidic channel;
exposing the particle to electromagnetic radiation and detecting an electromagnetic radiation signal; and
determining the density of the particle based upon the resonance frequency and the electromagnetic radiation signal.

2. A method for determining density of a particle, the method comprising:
flowing a plurality of particles through a microfluidic channel configured to receive the particle;
driving the microfluidic channel with an excitation element;
sensing a plurality of resonance frequencies of the suspended microchannel resonator as each of the particles flows through the suspended microchannel resonator;
exposing the plurality of particles to electromagnetic radiation and detecting an electromagnetic radiation signal for each of the particles; and
determining the density of each of the particles based upon at least a portion of the plurality of resonance frequencies,
wherein a coefficient of variation of an average of the densities is less than or equal to 1%.

3. The method of any one of the preceding claims, wherein the electromagnetic radiation signal comprises a difference between electromagnetic radiation detected in the presence of the particle and electromagnetic radiation detected in the absence of the particle under substantially identical conditions.

4. The method of any one of the preceding claims, further comprising sorting the particle based, at least in part, on the determining the density.

5. The method of any one of the preceding claims, wherein an average velocity of particles is greater than or equal to 0.05 mm/second and/or less than or equal to 100 mm/second.

6. A microfluidic system for determining a density of a particle, comprising:
a suspended microchannel resonator comprising a first microfluidic channel associated with an inlet;
an excitation element for driving the first microfluidic channel;
a second microfluidic channel in fluidic communication with the first microfluidic channel;
a source of electromagnetic radiation associated with the second microfluidic channel;
a sensor associated with the suspended microchannel resonator configured to sense a resonance frequency of the suspended microchannel resonator;
a detector configured to detect an electromagnetic radiation signal and associated with the second microfluidic channel; and
a processor for determining the density of a particle based upon the electromagnetic radiation signal and the resonance frequency of the microfluidic channel.

7. The system or method of any one of the preceding claims, wherein the particle is a single cell.

8. The system or method of any one of the preceding claims, wherein the electromagnetic radiation is light.

9. The system or method of any one of the preceding claims, wherein the density is resolved within at least 0.001 g/mL.

10. The system or method of any one of the preceding claims, wherein the first microfluidic channel has a cross-sectional dimension of greater than or equal to 10 μm and/or less than or equal to 1 mm.

11. A system or method of any one of the preceding claims, wherein the particle is suspended in a fluid.

12. The system or method of any one of the preceding claims, wherein the first microfluidic channel has a throughput of greater than or equal to 6,800 particles/min and less than or equal to 100,000 particles/min.
